(19) United States
(12) Patent Application Publication
(10) Pub. No.: US 2018/0161039 A1
(43) Pub. Date: Jun. 14, 2018

(54) IMPLANTABLE MEDICAL DEVICE AND SYSTEM TO HEAT TISSUE

(71) Applicant: KONINKLIJKE PHILIPS N.V., EINDHOVEN (NL)

(72) Inventor: Godefridus Antonius Harks, Best (DE)

(21) Appl. No.: 15/575,514

(22) PCT Filed: Jun. 12, 2016
(86) PCT No.: PCT/EP2016/063414

§ 371 Date: Nov. 20, 2017

(30) Foreign Application Priority Data
Jun. 19, 2015 (EP) 15172878.9

(51) Int. Cl. A61B 17/12; A61B 17/00

(52) U.S. Cl. CPC A61B 17/12122; A61B 17/1214; A61B 2018/00577; A61B 17/12109; A61B 17/0057

(57) ABSTRACT

A medical implant device (18) for atrial appendage (20) is presented, suitable to transmit energy to its surrounding. The medical implant device (18) comprising an expandable structure (31,34) and a cover (32) disposed on the expandable structure is adapted to extend across the ostium (21) of the atrial appendage (20) for restricting migration of thrombi from the appendage to the blood stream in the heart (13). The expandable structure (31,34) of the medical implant device (18) configured to receive energy from an external energy source (11) is further configured to transmit energy to its surrounding for altering the electrical activation pathways in heart by heating the tissue adjacent to the medical implant device (18).

### FIELD OF THE INVENTION

The present invention relates to a medical implant device comprising an expandable structure and a cover disposed on the expandable structure, adapted to extend across an ostium of an atrial appendage. The invention further relates to a system comprising the medical implant device and a method for using the system.

### BACKGROUND OF THE INVENTION

Atrial fibrillation is the most common form of irregular heartbeat. It is caused by abnormal propagation of electrical signals in the heart tissue. The most important consequence is blood stagnation in the left atrial appendage. The irregular topography of the inner surface of the left atrial appendage, formed by folds compartmented by muscular ridges, creates favorable conditions for creation of blood clots (thrombi). The thrombi may increase in size and eventually brake in fragments or escape from the place at which they originate. Fragments of thrombi reaching the blood stream through the left atrium, and being propelled in the circulatory system, are causing the vast majority of non-valvular atrial fibrillation related strokes.

Closure of the left atrial appendage may provide long-term protection by reducing stroke related conditions originating from atrial fibrillation. This can be achieved in a minimally invasive procedure that involves placement of a “plug” in the left atrial appendage.

WO2012109297 presents various atrial appendage implant devices adapted for use in therapy of cardiac arrhythmia of a patient. The implant device comprises an anchoring portion adapted to anchor the device in place, and a barrier element adapted to prevent blood clots passing through the barrier element. In a deployed state of the device, the barrier element covers the orifice of the atrial appendage and therewith the implant device prevents blood flow into the atrial appendage. In an embodiment the appendage implant device comprises beside the anchoring portion and the barrier element also an arrhythmia treatment element adapted to treat a detected cardiac arrhythmia. The anchoring portion formed by anchoring elements comprises electrodes adapted to monitor the cardiac activity. A system adapted to process the electrical activity data received from the cardiac tissue through the electrodes, is configured to detect occurrence of atrial fibrillation, an unorganized electrical activity of the heart. When atrial fibrillation is detected, the system is further adapted to provide electrical pacing therapy by delivering electrical impulses to the cardiac tissue through the electrodes integrated in the anchoring elements, upon which the normal activity of the heart may be restored.

In a different embodiment of the system, the implantable device comprises a cardiac monitor and a drug reservoir, both disposed on the appendage side of the implantable device. The cardiac monitor is adapted to release a prescribed amount of the therapeutic agent in the event that atrial fibrillation is detected which lasts longer than a prescribed period of time. The therapeutic agent includes anti-arrhythmic and/or anticoagulation drugs.

The appendage implant device attempts to restore the normal electrical activity of the heart by either stimulating the heart with electrical pacing impulses to overrule the erratic electrical activity of the heart, or by administering anti-arrhythmic drugs to regulate the electrical activity of the heart. Additionally, the appendage implant device may administer anticoagulation drugs to inhibit blood coagulation.

WO2013009872 presents a system comprising a portable control device, an interface communication unit and an occlusion device having a trasponder unit. The transponder unit is including a sensor stage and a treatment stage, wherein the treatment stage comprises an electrical pulsing stage used to apply an electrical pulse to the heart in response to a sensed atrial fibrillation condition or bradycardia condition such as may occur after conversion of AF to sinus rhythm sensed by the sensor stage, and wherein the treatment stage further comprises pharmaceutical agent release stage used to release a pharmaceutical agent into the left atrial appendage for treatment of AF or heart failure or otherwise as determined from the sensor data from the sensor stage.

### SUMMARY OF THE INVENTION

It is an object of the invention to improve the effectiveness of the implant device.

According to the invention, this object is realized by a medical implant device releasably attachable to a catheter, comprising:

an expandable structure,

a cover disposed on the expandable structure, adapted to extend across an ostium of an atrial appendage of a heart,

wherein the expandable structure is configured to receive energy from an external energy source and is further configured to transmit energy to its surrounding such as to heat heart tissue in its surrounding when the expandable structure is in an expanded state that causes permanent occlusion of the atrial appendage.

The medical implant device deployed in an appendage of the heart of a patient addresses the consequences of the condition arising from atrial fibrillation by restricting release of thrombi form the appendage into the circulatory system. This is achieved by the cover disposed on the expandable structure of the medical implant device. The heat transmitted to the surrounding of the implant device changes the properties of its surrounding. Since heart tissue is located in the surrounding of the medical implant device, the heat causes modification of the biophysical properties of this heart tissue, leading to alteration of the electrical activation pathways of the heart.

The source that induces atrial fibrillation can be a focus or a reentrant wave, and may act as a driver or a trigger of atrial fibrillation. In case of a trigger, its initial activity sets off as self-sustained multiple wavelet reentry in the rest of the atrium (substrate) and even if the trigger is eliminated, the episode of atrial fibrillation continues. Atrial fibrillation maintenance depends on the interplay between two factors: the incidence of activity of the trigger and the fertility of the substrate to independently sustain multiple wavelet reentry. The ability of maintaining atrial fibrillation depends on the size of the substrate, with a higher likelihood of maintaining atrial fibrillation for a larger substrate. Changing the electrical properties of the tissue locally around the ostium of the left atrial appendage upon transmission of energy to the tissue by the medical implant device results in electrical isolation of the left atrial appendage from the rest of the heart, thereby the dimension of the substrate for maintenance and perpetuation of atrial fibrillation significantly reduces. In addition, triggers originating from the left atrial appendage are isolated from the rest of the atrium.

In an embodiment of the medical implant device, the cover disposed on the expandable structure has a mesh structure. Alternatively, the cover may be made of biocompatible fabric. In yet a further embodiment the cover may be a balloon inflatable with fluid, for instance saline solution. The cover restricts migration of thrombi formed in the appendage, thereby avoiding thrombi being propelled in the circulatory system. The expandable structure of the medical implant device may be formed by a metallic wireframe, preferably from shape memory alloy.

The medical implant device may be configured to transmit energy to the surrounding at its circumference in multiple segments. In an embodiment, the medical implant device is configured to transmit energy to its surrounding around its circumference along a spatially continuous contour. Electric isolation of the appendage from the rest of the heart at the level of the ostium can be achieved either by transmitting energy to the tissue in a sequential manner by the segments, or by transmitting energy to the tissue simultaneously with all segments along the entire contour.

In an embodiment of the medical implant device the received energy by the device has the same form as the transmitted energy to the surrounding. Such embodiment may use energy in the form of laser radiation or energy in the form of radiofrequency current. The advantage of no energy transformation necessary within the medical implant device confers manufacturing simplicity.

In another embodiment, the medical implant device is configured to transform the received energy to a different form of energy, which is subsequently transmitted to the surrounding.

In an embodiment of the medical implant device the expandable structure is arranged to receive electric energy by means of an electric current, and is further arranged to transform the electric energy into heat transmitted to its surroundings.

In an alternative embodiment of the medical implant device the expandable structure is arranged to receive energy by means of mechanical waves in the form of pressure waves, and is further arranged to transform the energy into heat transmitted to its surrounding. Sound waves are the most common pressure waves, with great practicality of ultrasound waves. The transformation of the received energy in the expandable structure of the medical implant device is based on heating due to vibration and subsequent friction of atoms or molecules. The main advantage of such embodiment is that the energy source does not necessarily need to be mechanically connected to the medical implant device in order to transfer energy. Similarly, in an embodiment of the medical implant device wherein the expandable structure is arranged to receive electric energy by means of electromagnetic waves, and which is further arranged to transform it into heat transmitted to its surrounding, the energy source does not need to be mechanically connected to the medical implant device, since the electromagnetic waves can be transmitted by the media between the energy source and the medical implant device.

In yet a further embodiment of the medical implant device, the expandable structure comprises measurement sensors. The sensors can measure various parameters such as temperature and electrical signals characterizing the heated tissue, which may be used as feedback to control the amount of energy transmitted to the tissue.

In a further aspect of the invention a system is presented, comprising the medical implant device and an external energy source.

In an embodiment, the system further comprises a catheter connecting the medical implant device with the external energy source. The catheter may fulfill multiple functions, specifically delivering and deploying the medical implant device at the designated location and facilitating transmission of energy from the external energy source to the medical implant device. Transmission of energy through the catheter occurs through electrical wiring or optical fibers, depending on the form of energy released by the external energy source.

In a different embodiment, there is no mechanical connection between the medical implant device and the energy source. The medical implant device is navigated towards the designated location by a catheter and it is deployed at that site by the catheter, without the catheter further enabling energy transmission between the external energy source and the medical implant device. The transmission of energy between the external energy source and the medical implant device occurs through transmission of energy by the media among the energy source and the medical implant device. Most frequently the media is a combination of fluid and the body of the patient. The external energy source in such embodiments emits electromagnetic waves or pressure waves.

In yet another aspect of the invention a method is presented for heating heart tissue, the method comprising:

introducing a medical implant device according to the invention into the heart with a catheter,

deploying the medical implant device in the atrial appendage of the heart,

the medical implant device receiving energy from an external energy source,

the medical implant device transmitting energy to the heart tissue such as to heat the heart tissue when the expandable structure is in expanded state that causes permanent occlusion of the atrial appendage. Introduction of the medical implant device into the heart and deployment at the right position in the appendage of the heart is enabled by a catheter. The medical implant device may receive energy from the external energy source either through the catheter or transmitted through the body of the patient. By heating the heart tissue adjacent to the medical implant device, the appendage can be electrically isolated from the rest of the heart, thereby the dimension of the substrate for maintenance and perpetuation of atrial fibrillation can significantly be reduced in patients.

The method may further comprise:

measuring temperature and/or electrical signals of the heart tissue adjacent to sensors integrated into the medical implant device,

controlling the amount of energy transmitted from the external energy source to the medical implant device and/or an activation sequence of the segments around the circumference of the medical implant device by a controller computer, based on the measured temperature and/or electrical signals. The controller computer comprises a computer program that enables selection of parameters such as the amount of energy transmitted from the external energy source to the medical implant device, temperature distribution required by the surrounding of the medical implant device, or activation sequence of the segments around the circumference of the medical implant device.

Additional aspects and advantages of the invention will become more apparent from the following detailed description, which may be best understood with reference to and in conjunction with the accompanying drawings.

### BRIEF DESCRIPTION OF THE DRAWINGS

In the drawings:

FIG. 1 shows schematically and exemplarily a system for transmitting energy to the surrounding of the medical implant device according to the invention,

FIG. 2 illustrates the positioning and deploying of the medical implant device in the atrial appendage of the heart of a patient,

FIG. 3A shows a close up schematic representation of the positioning of the medical implant device in a collapsed mode into the appendage,

FIG. 3B shows a close up schematic representation of the deployed medical implant device into the appendage, and released from the catheter,

FIG. 4 shows schematically and exemplarily a medical implant device according to the invention,

FIGS. 5A and 5B show schematic and exemplary representations of various embodiments of the medical implant device according to the invention,

FIG. 6 shows schematically and exemplarily a further embodiment of the medical implant device according to the invention,

FIG. 7 shows schematically and exemplarily an embodiment of the medical implant device comprising an inflatable balloon, according to the invention,

FIG. 8 shows schematically and exemplarily the steps of a method for heating heart tissue, according to the invention.

### DETAILED DESCRIPTION OF EMBODIMENTS

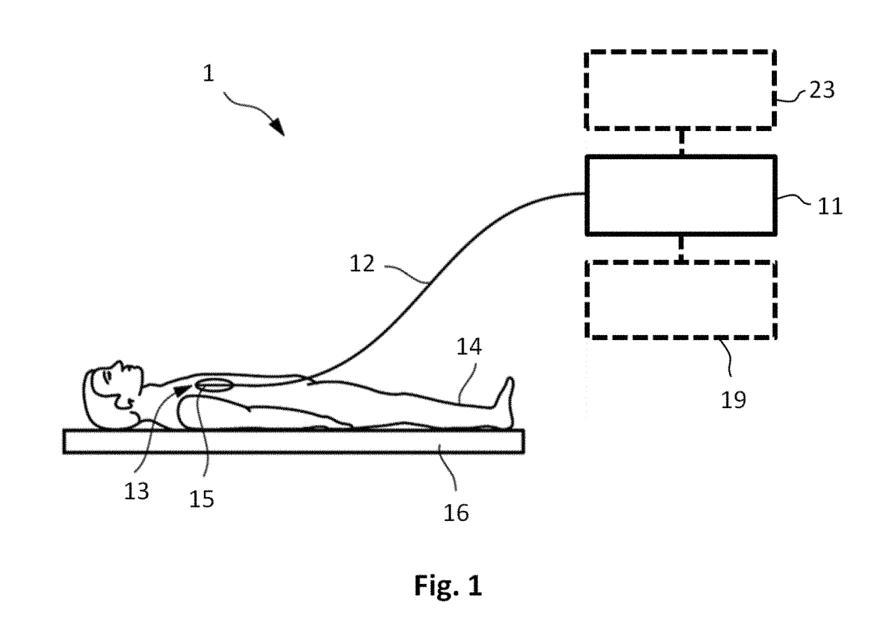

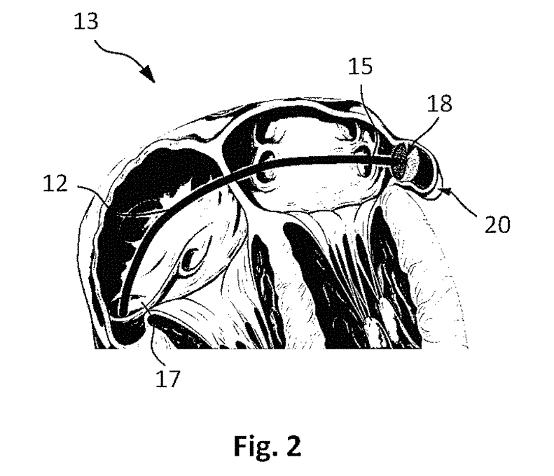

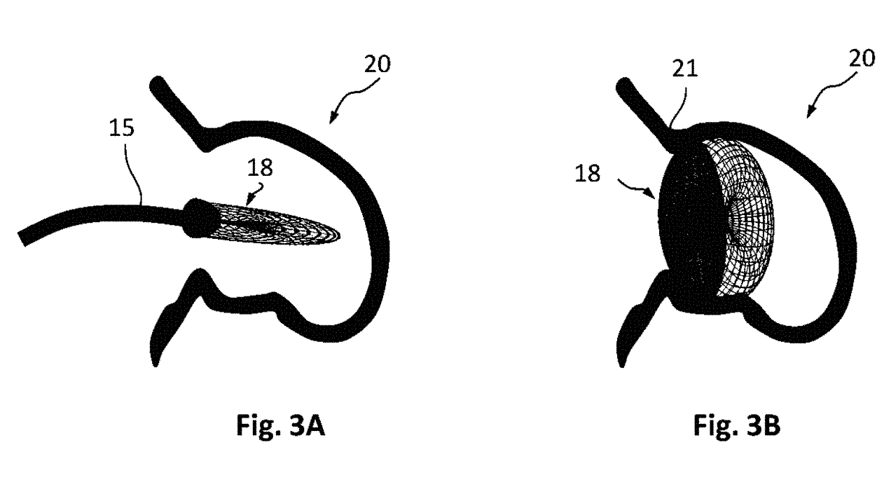

In FIG. 1 a system 1 is presented for introducing a medical implant device into the heart 13 of a patient 14 resting on a hospital bed 16. The system comprises an external energy source 11 connected to a catheter 12, a measurement apparatus 19, and a controller computer 23. The distal end 15 of the catheter 12 is introduced through a blood vessel 17 into the heart 13, as shown in FIG. 2. A medical implant device 18 is connected to the distal end 15 of the catheter, which is navigated towards the atrial appendage 20 in a collapsed mode, as schematically presented in FIG. 3A. Upon reaching the designated position in the atrial appendage, the medical implant device 18 is released from the catheter and it expands such that it forms a barrier between the atrial appendage 20 and the rest of the heart at the level of the ostium 21, as illustrated in FIG. 3B. Fluoroscopic imaging, ultrasound or magnetic resonance imaging may support the navigation of the medical implant device to the designated location.

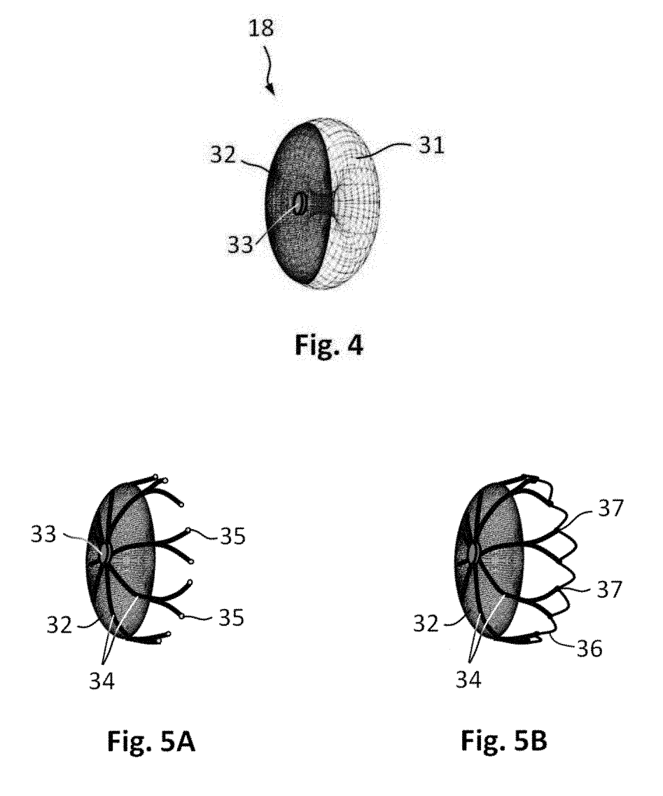

FIG. 4 shows a schematic representation of an exemplary embodiment of the medical implant device according to the invention. The medical implant device comprises an expandable structure 31, a cover 32 disposed on the expandable structure and a connection plug 33. The expandable structure 31 may be formed from a metallic wireframe. Shape memory alloy (e.g. nitinol) is an optimal choice of material, since the expandable structure may be given a predetermined shape by a heat treatment, and upon release of the medical implant device from the catheter at the designated location in atrial appendage, the expandable structure attempts to regain its predetermined shape, pressing against the tissue of the appendage, and thereby occluding the atrial appendage by permanently remaining at the deployment location after expansion. The cover 32 has a mesh structure. The mesh structure may be formed by densely packed metallic wireframe from similar material than that of the expandable structure, or from a different metal or alloy. The role of the cover 32 is to restrict migration of thrombi formed in the appendage due to atrial fibrillation, thereby avoiding thrombi being propelled in the circulatory system. The plug 33 enables connection of the medical implant device to the catheter for navigation purposes, as well as for transmission of energy from the external energy source 11 to the medical implant device 18 and for transmission of measurement signals (e.g. temperature, electrograms) from the medical implant device to the measurement apparatus 19. The expandable structure 31 is formed by segments of metallic wires, which receive energy from the external energy source. The received energy may be transferred to the heart tissue adjacent to the medical implant device in the same energy form. This can be the case when segments of the expandable structure receive the energy in the form of electric energy by means of a radio frequency current. In order to heat the appendage tissue adjacent the medical implant device, the medical implant device may be configured to transmit the electric energy in a bipolar or in a unipolar manner. Bipolar radiofrequency ablation occurs when the radiofrequency current is flowing through the heart tissue located between two segments of the expandable structure, thereby causing Joule heating of the tissue. For unipolar radio frequency ablation the radiofrequency current flows through the tissue located between a segment of the expandable structure and a neutral electrode (not shown) fixed on the body of the patient. The effect of Joule heating occurs at the site where the surface of the electrode in contact with the tissue is smaller, which is at the site adjacent to the segment of the expandable structure of the medical implant device, since the neutral electrode on the body of the patient is a patch of a considerable size. The medical implant device may be configured to transmit energy to the surrounding at its circumference in multiple segments, such that the transmission of energy from the medical implant device to the appendage tissue occurs around its circumference along a spatially continuous contour. The heating of the tissue by either bipolar or unipolar radio frequency ablation is preferentially at a temperature higher than 50 degrees Celsius, in order to cause protein denaturation in the tissue, which changes the electrical property of the tissue from electrically conductive to electrically isolating. Electric isolation of the appendage from the rest of the heart at the level of the ostium can be achieved either by transmitting energy to the tissue in a sequential manner by the segments, or by transmitting energy to the tissue simultaneously with all segments around the entire contour. Sensors may be embedded into the segments of the expandable structure 31 for a good control of the temperature and for measurement of electrical signals from the tissue adjacent to the medical implant device. Temperatures higher than 85 degrees Celsius within the tissue should be avoided during the ablation process for an optimal thermal treatment. Local boiling of the water content of the tissue due to excessively high temperature results in formation of steam pocket and eventual tissue rupture.

In a different embodiment of the system 1, the external energy source 11 transmits electromagnetic energy from outside of the body of the patient. The transformation of the received energy in the expandable structure 31 of the medical implant device 18 is based on inductive heating of the metallic expandable wireframe. The main advantage of such embodiment is that the energy source does not necessarily need to be mechanically connected to the medical implant device in order to transfer energy.

In alternative embodiments of the medical implant device presented in FIGS. 5A and 5B, the segments of the expandable structure 34 comprise optical fibers and they receive energy in the form of laser radiation from the external energy source 11 through the catheter 12 and the plug 33. The optical fibers may have terminations 35 at the ends of the segments of the expandable structure 34, such that the transfer of laser radiation to the surrounding occurs directly in the same form of energy as received by the medical implant device. Alternatively, the termination 35 of the optical fibers may be directed towards a structure 36 in the joint points 37. The material of the structure 36 is made of laser radiation absorbent material, such that the received energy is transformed into heat at the joints 37. The structure 36, segmented by the joints 37, may be considered as part of the expandable structure 34. The segments 36 are preferably made of heat conductive material, such that the segments 36 heat up on their entire length starting from the joints 37. In such way the expandable structure can be configured to transmit energy to the heart tissue in the form of heat, around the circumference of the expandable structure and along a spatially continuous contour, causing change in the electrical property of the tissue locally around the ostium of the atrial appendage, thereby forming two domains of electrically conductive tissue separated by an electrically insulating one.

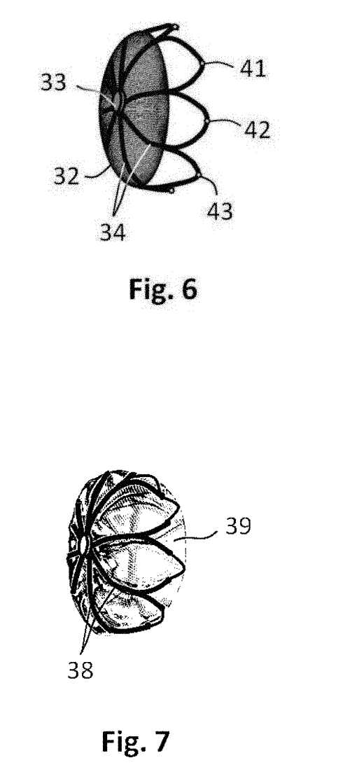

In an embodiment of the medical implant device presented in FIG. 6, the expandable structure 34 comprises sensors 41,42,43 for measuring temperature of the tissue during transfer of energy from the medical implant device to the tissue. Instead of measuring temperature, the sensors may be configured to measure electrical signals originating from the tissue. The sensors allow a better control of the energy transfer to the tissue, such that the required effect of the energy transfer occurs. The feedback for the energy control can be based on temperature measurement or on the magnitude of the electrical signals originating from the heart tissue and indicating local electrical activation. The measurement signals may be transferred to the measurement apparatus 19 through wired connection via the catheter 12 or through wireless connection. In case of wireless connection, the transmission means are embedded in an extension of the plug 33.

In a different embodiment of the medical implant device, the expandable structure 34 may be made of an electrically insulating material, and the sensors 41,42,43 may be used as electrodes for transferring energy by means of radio frequency current to the tissue, either in bipolar or in a unipolar configuration. The transfer of energy to the tissue may be performed intermittently, such that in the short interruption intervals electrical signals originating from the heart tissue can be measured and acquired.

The cover in the form of an inflatable balloon 39 may be disposed on a metallic wireframe 38 of the expandable structure from the inside, as illustrated in FIG. 7. The balloon restricts migration of thrombi from the atrial appendage to the circulatory system when inflated with fluid, for instance with saline solution. The metallic wireframe 38 must not necessarily be made of shape memory alloy, since the pressure that is exerted by the balloon when inflated with saline presses the metallic wireframe in contact with the tissue of the atrial appendage. The balloon may remain inflated with saline after deployment of the medical implant device, since even when saline escapes from the balloon due to the finite permeability of the balloon material, the saline is tolerated by the organism. After a period, the blood pool in the atrial appendage proximal to the medical implant device coagulates and the balloon structure 39 effectively stops migration of coagulum fragments into the circulatory system.

The balloon 39 can be made of polyethylene terephthalate. In an embodiment, the polyethylene terephthalate may further be covered by an electrically conductive material, preferably a thin metal layer deposited either by evaporation or sputtering. The metallic layer may be used as electrode to transfer energy to the tissue by means of radio frequency current. In an embodiment of the medical implant device, the combination of fluid inside the balloon, the material of the balloon and the coating on the balloon have properties to generate heat when the expandable structure is arranged to receive energy by means of mechanical waves in the form of pressure waves (e.g. sound waves) from the external energy source 11. In such embodiment of the system, the external energy source 11 does not need to be mechanically connected to the medical implant device 18, since sound waves can easily be transmitted through the body of the patient. The transformation of the received energy by the medical implant device is based on heating of the expandable structure due to vibration of the atoms or molecules upon the pressure waves and subsequent friction between atoms or molecules, or friction of the atoms or molecules at the various interfaces in the composition of the medical implant device. The generated heat is transmitted to the tissue and it alters the electrical conductivity of the tissue, thereby creating an electrical insulation region between the atrial appendage and the atrium at the location of deployment of the medical implant device.

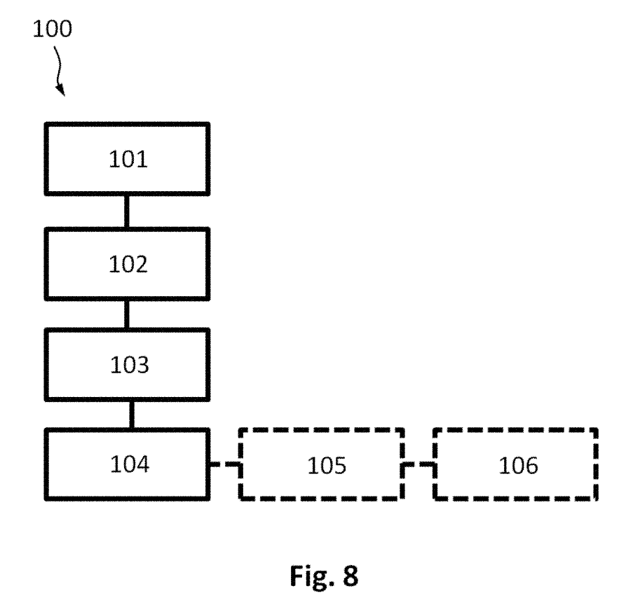

FIG. 8 shows a schematic representation of a method 100 for heating heart tissue with the medical implant device, according to the invention. The method comprises the following steps: step 101 in which the medical implant device 18 is introduced in collapsed mode through the blood vessel 17 into the heart 13 of a patient 14; step 102 wherein the medical implant device is deployed in the atrial appendage of the heart; step 103 wherein the medical implant device 18 receives energy from the external energy source 11; and step 104 wherein the medical implant device 18 transmits energy to the heart tissue such as to heat the heart tissue. Introduction of the medical implant device into the heart and deployment at the right position in the appendage of the heart is enabled by the catheter 12. The medical implant device 18 may receive energy from the external energy source 11 through the catheter before the release of the medical implant device from the catheter. In other embodiments the external energy source 11 transmits energy to the medical implant device 18 through the body of the patient. The medical implant device may transmit the received energy to the tissue either in the form in which it has received it from the external energy source, or in other form of energy. The tissue is heated by the medical implant device to a temperature above 50 degrees Celsius, in order to alter the electrical conductivity of the adjacent tissue, resulting in a separation of the electrical activity of the rest of the heart from the appendage tissue.

The system 1 may be controlled from a computer 23, which can either be connected to the external energy source 11 or it can be integrated in the external energy source module. The controller computer 23 comprises a program reader which can read a computer-readable medium having stored a computer-executable program. The computer-executable program comprises program code means for causing the medical implant device 18 to receive energy from the external energy source 11 and to transmit energy to its surrounding when the computer-executable program is run on the computer controlling the system 1. The controller computer 23 may receive measurement signals from the measurement apparatus 19, and it can be configured to analyze the measurement signals. The computer-executable program may use the information to adjust the amount of energy transmitted from the external energy source 11 to the medical implant device 18, such that the temperature of the tissue adjacent to the measurement sensors 41,42,43 does not exceed 85 degrees Celsius when the medical implant device 18 transmits energy to the tissue. Analogously, the energy transmitted from the external energy source 11 to the medical implant device 18 may be adjusted based on the magnitude of the electrical signals measured by the measurement sensors 41,42,43. Additionally, the computer program may enable selection of parameters such as the amount of energy transmitted from the external energy source to the medical implant device, temperature distribution required by the surrounding of the medical implant device, or activation sequence of the segments around the circumference of the medical implant device. An alternative to reduction of the amount of energy transmitted from the external energy source to the medical implant device in order to limit the temperature of the tissue not to exceed the upper threshold limit is activation of the segments in a predefined sequence. This may be obtained by assigning duty time for particular segments during which transfer of energy occurs from the respective segment to the tissue and/or assigning repetitive activation of certain segments, based on local thickness of the adjacent tissue.

The control of the parameters such as the amount of energy transmitted from the external energy source 11 to the medical implant device 18 and the activation sequence of the segments 31,34,36 may be rendered as additional steps of the method 100, schematically illustrated in FIG. 8 with dashed line. Step 105 represents temperature measurement and/or measurement of electrical signals of the heart tissue adjacent to the sensors 41,42,43 integrated into the medical implant device 18. In step 106 the controller computer 23 regulates the amount of energy transmitted by the external energy source 11 to the medical implant device 18 and/or the activation sequence of the segments of the expandable structure 31,34,36 around the circumference of the medical implant device, based on the measured temperature and/or electrical signals.

Although the medical implant device was used in the exemplary description of the invention, that should not be construed as limiting the scope.

Other variations to the disclosed embodiments can be understood and effected by those skilled in the art in practicing the claimed invention, from a study of the drawings, the disclosure, and the appended claims.

A single unit or device may fulfill the functions of several items recited in the claims. The mere fact that certain measures are recited in mutually different dependent claims does not indicate that a combination of these measures cannot be used to advantage.

In the claims, the word “comprising” does not exclude other elements or steps, and the indefinite article “a” or “an” does not exclude a plurality.

Any reference signs in the claims should not be construed as limiting the scope.

1. A medical implant device releasably attachable to a catheter, comprising:
an expandable structure,
a cover disposed on the expandable structure, adapted to extend across an ostium of an atrial appendage of a heart,
wherein the expandable structure is configured to receive energy from an external energy source and is further configured to transmit energy to its surrounding such as to heat heart tissue in its surrounding to change an electrical property of the heart tissue from electrically conductive to electrically isolating when the expandable structure is in an expanded state that causes permanent occlusion of the atrial appendage by the medical implant device.

2. The medical implant device according to claim 1, wherein the expandable structure is made of shape memory alloy.

3. The medical implant device according to claim 1, wherein the expandable structure is configured to transfer the received energy into transmitted energy of a different form.

4. The medical implant device according to claim 1, wherein the expandable structure is arranged to receive the energy in the form of electric energy by means of a radiofrequency current provided to the expandable structure.

5. The medical implant device according to claim 1, wherein the expandable structure is arranged to receive the energy in the form of laser radiation.

6. The medical implant device according to claim 3, wherein the expandable structure is arranged to receive electric energy by means of an electric current provided to the expandable structure, and further arranged to transform it into heat transmitted to its surroundings.

7. The medical implant device according to claim 3, the expandable structure arranged to receive electric energy by means of electromagnetic waves, and further arranged to transform it into heat transmitted to its surrounding.

8. The medical implant device according to claim 3, the expandable structure arranged to receive energy by means of mechanical waves in the form of pressure waves, and further arranged to transform it into heat transmitted to its surrounding.

9. The medical implant device according to claim 1, wherein the cover is a balloon that is inflatable with fluid.

10. The medical implant device according to claim 1, wherein the expandable structure comprises measurement sensors.

11. The medical implant device according to claim 1, configured to transmit energy to the surrounding around its circumference along a spatially continuous contour.

12. A system comprising the medical implant device according to claim 1 and an external energy source for providing the energy to be received by the expandable structure.

13. The system according to claim 12, further comprising a catheter for connecting the medical implant device to the external energy source.

14. A method of heating heart tissue with the medical implant device according to claim 1, the method comprising:
introducing the medical implant device into the heart with a catheter,
deploying the medical implant device in the atrial appendage of the heart,
the medical implant device receiving energy from an external energy source,
the medical implant device transmitting energy to the heart tissue such as to heat the heart tissue in its surrounding to change an electrical property of the heart tissue from electrically conductive to electrically isolating when the expandable structure is in expanded state that causes permanent occlusion of the atrial appendage by the deployed medical implant device.

15. The method according to claim 14, the method further comprising:
measuring temperature and/or electrical signals of the heart tissue adjacent to sensors integrated into the medical implant device,
controlling an amount of energy transmitted from the external energy source to the medical implant device and/or an activation sequence of segments of the expandable structure around the circumference of the medical implant device by a controller computer, based on the measured temperature and/or electrical signals.
